(19) United States
(12) Patent Application Publication
(10) Pub. No.: US 2023/0236146 A1
(43) Pub. Date: Jul. 27, 2023

(54) GAS SENSOR

(71) Applicant: DENSO CORPORATION, Kariya-city (JP)

(72) Inventor: Daisuke MIZUNO, Kariya-city (JP)

(21) Appl. No.: 18/194,269

(22) Filed: Mar. 31, 2023

Related U.S. Application Data
(63) Continuation of application No. PCT/JP2021/033660, filed on Sep. 14, 2021.

(30) Foreign Application Priority Data
Oct. 2, 2020 (JP) 2020-167905

(51) Int. Cl. G01N 27/406; G01N 27/419; G01N 27/41

(52) U.S. Cl. CPC G01N 27/4067; G01N 27/419; G01N 27/41; G01N 33/0037

(57) ABSTRACT

A gas sensor includes a detection circuit unit that detects a specific gas component in measured gas based on output from a sensor element. The detection circuit unit includes an AC voltage application unit that applies an AC voltage signal to a pair of electrode units in an electrochemical cell, a gas concentration detection unit that detects concentration information on the specific gas component from a DC signal component included in an output signal provided by the electrochemical cell, and a cell temperature detection unit that detects temperature information on the electrochemical cell from an AC signal component included in the output signal. The cell temperature detection unit includes a signal extraction unit that removes the DC signal component to separate the AC signal component from the output signal, and a synchronous detection unit that performs synchronous detection on the separated AC signal component using the AC voltage signal.

### CROSS-REFERENCE TO RELATED APPLICATION

The present application is a continuation application of International Application No. PCT/JP2021/033660, filed on Sep. 14, 2021, which claims priority to Japanese Patent Application No. 2020-167905, filed on Oct. 2, 2020. The contents of these applications are incorporated herein by reference in their entirety.

### BACKGROUND

### Technical Field

The present disclosure relates to a gas sensor that detects the concentration of a specific gas in a measured gas.

### Background Art

A gas sensor is placed in an exhaust gas passage for an internal combustion engine so as to detect the concentrations of various gases in exhaust gas. Such a gas sensor is used as a sensor such as an air-fuel ratio sensor and/or a NOx sensor in order to monitor the combustion state of the internal combustion engine or the operation of an exhaust gas processing device, and typically includes a solid electrolyte sensor element. The solid electrolyte sensor element includes an electrochemical cell having a pair of electrodes on the surface of a solid electrolyte layer conducting oxide ions and may have an element structure or a detection scheme appropriate to the type of gas to be measured.

### SUMMARY

In the present disclosure, provided is a gas sensor as the following.

The gas sensor includes a detection circuit unit that detects a specific gas component in measured gas based on output from a sensor element. The detection circuit unit includes an AC voltage application unit that applies an AC voltage signal to a pair of electrode units in an electrochemical cell, a gas concentration detection unit including an averaging processing unit that averages an output signal provided by the electrochemical cell to extract a DC signal component included in the output signal, and detects concentration information on the specific gas component from the DC signal component, and a cell temperature detection unit that detects temperature information on the electrochemical cell from an AC signal component included in the output signal. The cell temperature detection unit includes a signal extraction unit including a subtraction processing unit that subtracts the extracted DC signal component from the output signal to extract the AC signal component, and a synchronous detection unit that performs synchronous detection on the separated AC signal component using the AC voltage signal. The synchronous detection unit includes a multiplication processing unit that multiplies the extracted AC signal component by the applied AC voltage signal, and a filter unit that filters the signal after the multiplication to allow passage of components with frequencies lower than a frequency of the applied AC voltage signal.

### BRIEF DESCRIPTION OF THE DRAWINGS

The above and other objectives, features, and advantages of the present disclosure will be clearly apparent from the detailed description provided below with reference to the accompanying drawings, in which:

FIG. 1 is a schematic configuration diagram including a sensor element and a detection circuit unit that serve as the main part of a gas sensor according to a first embodiment;

FIG. 2 is a diagram illustrating the overall configuration of the gas sensor according to the first embodiment;

FIG. 3 is a cross-sectional view in the longitudinal direction of the sensor element illustrating its distal end configuration according to the first embodiment;

FIG. 4 is a cross-sectional view in the width direction of the sensor element illustrating the distal end configuration of the element according to the first embodiment;

FIG. 5 is a block diagram illustrating an example of the configuration of an AC voltage generation unit in the detection circuit unit according to the first embodiment;

FIG. 6 is a diagram illustrating the relationship between cell impedance and the frequency of AC voltage applied to the sensor element according to the first embodiment;

FIG. 7 is a diagram illustrating various signals separated from signals output to the detection circuit unit and signals applied to the sensor element, and the waveforms of the signals according to the first embodiment;

FIG. 8 is a diagram illustrating the relationship between signals and noise components after synchronous detection performed by a cell temperature detection unit in the detection circuit unit according to the first embodiment;

FIG. 9 is a diagram illustrating the relationship between signals and noise components before synchronous detection performed by the cell temperature detection unit in the detection circuit unit according to the first embodiment;

FIG. 10 is a diagram illustrating the relationship between the waveform of voltage applied to and the waveform of current output from the sensor element, comparing the first embodiment with a conventional example in which applied voltages are switched;

FIG. 11 is a diagram illustrating an example configuration of the detection circuit unit in which the main part is an analog arithmetic circuit and an example configuration in which the main part is a digital arithmetic circuit according to the first embodiment;

FIG. 12 is a block diagram illustrating another example of the configuration of the AC voltage generation unit in the detection circuit unit according to the first embodiment;

FIG. 13 is a diagram illustrating the overall configuration of a gas sensor according to a second embodiment; and

FIG. 14 is a cross-sectional view in the longitudinal direction of a sensor element illustrating its distal end configuration according to the second embodiment.

### DETAILED DESCRIPTION OF THE PREFERRED EMBODIMENTS

For example, a NOx sensor has an element configuration with a combination of multiple electrochemical cells and also functions as an air-fuel ratio sensor. Specifically, such a sensor is formed as a limiting current sensor element including a pump cell that pumps oxygen contained in measured gas introduced into the element through a diffusion resistor, and a sensor cell that detects the concentration of NOx in the measured gas after the pumping. Based on the fact that the current flowing in this pump cell is a limiting current depending on the oxygen concentration, the air-fuel ratio (i.e., A/F) of the internal combustion engine can be monitored.

The sensor element also incorporates a heater, and the energization of the heater is controlled to achieve a temperature appropriate to the operation of the electrochemical cells. Temperature detection for the sensor element is, for example, based on the correlation between the element temperature and the heater resistance or the impedance of the solid electrolyte layer, and the temperature of the sensor element can be detected without a separate temperature detecting element. For example, Patent Literature 1 describes a temperature measurement method implemented for a limiting current sensor that provides output depending on the oxygen level in response to power supply from a DC voltage source to the two electrodes of the sensor, the method using, as a measure of temperature, an alternating current flowing in response to power supply from an AC voltage source.

Specifically, the method described in Patent Literature 1 superimposes the output voltage of the AC voltage source onto that of the DC voltage source and separates signals extracted by a current measuring resistor through a high-pass filter and a low-pass filter. This high-pass filter is designed to filter out DC voltage components, and allows temperature-dependent AC voltage signals to be output. In contrast, the low-pass filter is designed to filter out the frequency of the AC voltage source, and allows DC voltage signals proportional to the oxygen concentration to be output.
[PTL 1] JP H04-24657 A

To further improve the efficiency of exhaust gas purification, gas sensors have recently been needed to detect a gas concentration with increased accuracy. In addition, sensor output has a dependence on temperature, and thus it is desirable to detect the temperature of the sensor element with high accuracy and reflect the detected temperature in, for example, heater control. In this detection, the impedance of the electrochemical cell may be used to detect temperature information on a site nearer a detection unit. However, the temperature is detectable while the gas concentration detection is stopped, and continuous detection cannot be performed. Additionally, as the number of on-vehicle electronic devices increases, the effect of high frequency noise caused under vehicle driving environments has become significant in the processing of detected signals.

As described in Patent Literature 1, a signal corresponding to the gas concentration can be extracted together with a signal that is a measure of temperature by using two filters to separate signals detected in response to the application of an AC voltage. However, the method extracts an AC signal as a measure of temperature using a high-pass filter or a band-pass filter that allows high frequency signals through and cannot eliminate noise components such as high frequency noise, degrading AC signal detection accuracy. Thus, gas concentration detection by applying a DC voltage and temperature detection by applying an AC voltage are conventionally performed at different times, and it is desired to achieve both a reduction in vehicle noise and continuous detection of the gas concentration and the temperature.

An objective of the present disclosure is to provide a gas sensor that uses a sensor element including an electrochemical cell to enable gas concentration information and temperature information to be simultaneously detected with high noise resistance and high detection accuracy.

An aspect of the present disclosure is a gas sensor comprising: a sensor element; and a detection circuit unit configured to detect a specific gas component in measured gas based on output from the sensor element,

wherein the sensor element includes
a measured gas chamber configured to receive the measured gas through a diffusion resistance layer, and
an electrochemical cell including a pair of electrode units respectively provided on a first surface and a second surface of a solid electrolyte layer, the first surface being in contact with the measured gas and the second surface being in contact with reference gas,

the detection circuit unit includes
an AC voltage application unit configured to apply an AC voltage signal to the electrochemical cell,
a gas concentration detection unit including an averaging processing unit configured to average an output signal provided by the electrochemical cell to extract a DC signal component included in the output signal provided by the electrochemical cell, and configured to detect concentration information on the specific gas component from the DC signal component, and
a cell temperature detection unit configured to detect temperature information on the electrochemical cell from an AC signal component included in the output signal provided by the electrochemical cell, and

the cell temperature detection unit includes
a signal extraction unit including a subtraction processing unit configured to subtract the extracted DC signal component from the output signal provided by the electrochemical cell to extract the AC signal component, and
a synchronous detection unit configured to perform synchronous detection on the extracted AC signal component using the AC voltage signal, and

the synchronous detection unit includes a multiplication processing unit configured to multiply the extracted AC signal component by the applied AC voltage signal, and a filter unit configured to filter the signal after the multiplication to allow passage of components with frequencies lower than a frequency of the applied AC voltage signal.

In the gas sensor having the above configuration, when the AC voltage application unit of the detection circuit unit applies an AC voltage signal to the electrochemical cell of the sensor element, the electrochemical cell provides an output signal including a DC signal component corresponding to the concentration information on the specific gas component and an AC signal component corresponding to the temperature information on the electrochemical cell. Thus, by separating the DC signal component from the output signal, the gas concentration detection unit obtains the concentration information on the specific gas component. In addition, by removing the DC signal component separated from the output signal, the cell temperature detection unit can extract the AC signal component. By subjecting the AC signal component to synchronous detection using the applied AC voltage signal, the DC component including the temperature information and an AC component are obtained, and thus this AC component and also a noise component can be removed to extract only the DC component including the temperature information.

As described above, the above aspect provides a gas sensor that uses a sensor element including an electrochemical cell to enable gas concentration information and temperature information to be simultaneously detected with high noise resistance and high detection accuracy.

### First Embodiment

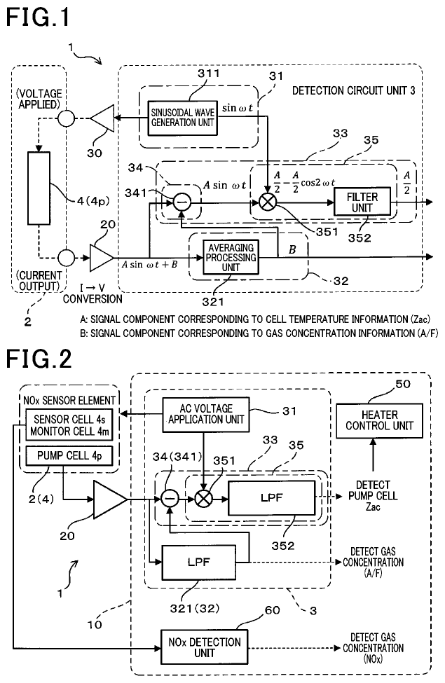

A gas sensor according to a first embodiment will now be described with reference to FIGS. 1 to 12.

In FIGS. 1 and 2, a gas sensor 1 according to the present embodiment is, for example, used in an exhaust gas purification system for a vehicle engine that is an internal combustion engine and detects the concentration of a specific gas in exhaust gas that is measured gas. More specifically, examples of the specific gas in the measured gas include various gas components such as oxygen and NOx. As shown in FIG. 2, the gas sensor 1 includes, for example, a sensor element 2 capable of detecting an oxygen concentration and a NOx concentration.

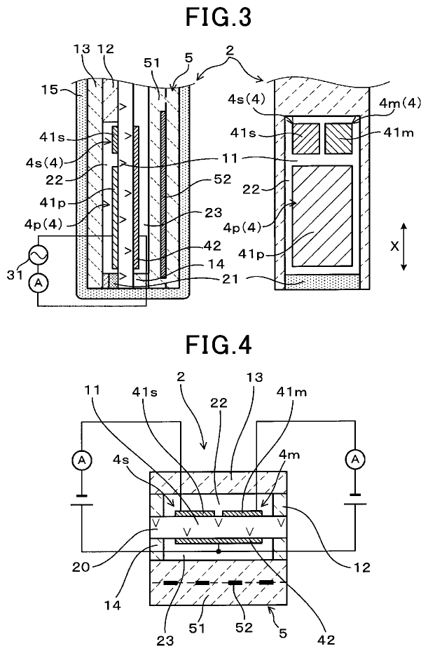

The gas sensor 1 includes the sensor element 2 and a detection circuit unit 3 that detects a specific gas component in measured gas based on output from the sensor element 2. The sensor element 2 includes one or more electrochemical cells 4. As shown in FIGS. 3 and 4, the sensor element 2 is designed as a limiting current sensor element and includes a measured gas chamber 22 that receives the measured gas through a diffusion resistance layer 21. Each electrochemical cell 4 includes a solid electrolyte layer 11 and a pair of electrode units 41, 42 provided on the solid electrolyte layer surface in contact with the measured gas and the solid electrolyte layer surface in contact with reference gas.

As shown in FIG. 1, the detection circuit unit 3 includes an AC voltage application unit 31, a gas concentration detection unit 32, and a cell temperature detection unit 33. The AC voltage application unit 31 applies an AC voltage signal (e.g., sin ωt) to the pair of electrode units 41, 42 of the electrochemical cell 4. The gas concentration detection unit 32 detects concentration information on the specific gas component (e.g., air-fuel ratio A/F; hereinafter referred to as gas concentration information) from an output signal (e.g., A sin ωt+B) provided by the electrochemical cell 4. In addition, the cell temperature detection unit 33 detects temperature information on the electrochemical cell 4 (e.g., cell impedance Zac; hereinafter referred to as cell temperature information) from the output signal provided by the electrochemical cell 4.

The cell temperature detection unit 33 also includes a signal extraction unit 34 that extracts an AC signal component (e.g., A sin ωt) from the output signal provided by the electrochemical cell 4 and a synchronous detection unit 35 that performs synchronous detection on the extracted AC signal component (e.g., A sin ωt) with the AC voltage signal (e.g., sin ωt) applied to the electrochemical cell 4.

Specifically, the gas concentration detection unit 32 includes an averaging processing unit 321 that averages the output signal provided by the electrochemical cell 4 to extract a DC signal component (e.g., B). The signal extraction unit 34 includes a subtraction processing unit 341 that subtracts the extracted DC signal component from the output signal provided by the electrochemical cell 4 to extract an AC signal component. The synchronous detection unit 35 includes a multiplication processing unit 351 that multiplies the extracted AC signal component by the applied AC voltage signal, and a filter unit 352 that filters the signal after the multiplication processing to allow the passage of components with frequencies lower than the frequency of the applied AC voltage signal.

This configuration enables the acquisition of a signal corresponding to gas concentration information by separating a DC signal component from the output signal provided by the electrochemical cell 4. Simultaneously, the separated DC signal component may be used to separate an AC signal component including cell temperature information. Furthermore, this AC signal component may be subjected to synchronous detection and then filtered to accurately detect the cell temperature information with high frequency noise components eliminated.

Suitably, the AC voltage application unit 31 includes an AC voltage generation unit (e.g., a sinusoidal wave generation unit 311) that generates a sinusoidal wave signal or a rectangular wave signal as the AC voltage signal, and continuously applies the AC voltage signal. The sensor element 2 may include multiple electrochemical cells 4. In this case, the detection circuit unit 3 is provided in correspondence with one or more of the electrochemical cells 4.

As shown in FIG. 2, suitably, the gas sensor 1 further includes a heater control unit 50 that controls the operation of a heater unit 5 incorporated in the sensor element 2. The heater control unit 50 performs feedback control of the temperature of the sensor element 2 based on the detection results from the cell temperature detection unit 33 included in the one or more detection circuit units 3.

With the gas sensor 1 having the configuration described above, the electrochemical cell 4 connected to the detection circuit unit 3 can simultaneously obtain gas concentration information and cell temperature information. In addition, noise components can be eliminated from the cell temperature information. These advantages provide the high-performance gas sensor 1 in which the sensor element 2 achieves both continuous gas concentration detection and temperature control.

### (Overall Configuration of Gas Sensor 1)

An example of the configuration of the gas sensor 1 will now be described in detail. As shown in FIG. 1, the gas sensor 1 includes the sensor element 2 including the one or more electrochemical cells 4 and the detection circuit unit 3 connected to the sensor element 2. The gas sensor 1 is a limiting current sensor that performs detection based on limiting currents flowing in the electrochemical cell 4. As illustrated in FIG. 2, the sensor element 2 has a triple-cell element structure including a pump cell 4p, a monitor cell 4m, and a sensor cell 4s, and the operation of the sensor element 2 is controlled by a sensor control unit 10 including the detection circuit unit 3.

The sensor control unit 10 includes the detection circuit unit 3 connected to at least one or two or more of the electrochemical cells 4, and the detection circuit unit 3 can simultaneously detect the gas concentration of a specific gas component and the cell temperature for the corresponding electrochemical cell 4. In the sensor element 2 shown in FIG. 2, for example, the pump cell 4p is a cell that detects the oxygen concentration (air-fuel ratio, A/F) simultaneously with the cell temperature that is a representative temperature of the sensor element 2 (hereinafter also referred to as an element temperature). The output from the pump cell 4p of the sensor element 2 is, for example, taken in the detection circuit unit 3 through a current-voltage conversion unit 20 including an amplifier.

The detection circuit unit 3 includes the AC voltage application unit 31, and the gas concentration detection unit 32 and the cell temperature detection unit 33, which receive and output signals to and from the pump cell 4p of the sensor element 2 and detect a gas concentration and a cell temperature. The sensor control unit 10, for example, controls the operation of the sensor element 2 in accordance with commands from a controller (not shown) for the vehicle engine (hereinafter referred to as an engine ECU). The gas concentration or other detection results from the sensor element 2 are output from the sensor control unit 10 to the engine ECU and, for example, used for control of the exhaust gas purification system including the gas sensor 1.

In the present embodiment, the sensor element 2 is, for example, designed as a NOx sensor element. The sensor element 2 discharges oxygen in exhaust gas through oxygen pumping by the pump cell 4p to adjust the oxygen concentration, and detects the air-fuel ratio A/F based on the current flowing as a result of the discharge. With the oxygen concentration adjusted, the monitor cell 4m monitors the concentration of the residual oxygen in the exhaust gas, and the effect of the residual oxygen can be eliminated from the output from the sensor cell 4s to detect the NOx concentration of the exhaust gas.

The sensor control unit 10 includes the heater control unit 50 and a NOx detection unit 60 in addition to the detection circuit unit 3. On the basis of, for example, the cell temperature detection results from the detection circuit unit 3, the heater control unit 50 performs feedback control of the operation of the heater unit 5 incorporated in the sensor element 2 so that the sensor element 2 is in a state appropriate to gas concentration detection. The NOx detection unit 60 can detect the concentration of NOx in the exhaust gas on the basis of, for example, a difference between outputs from the sensor cell 4s and the monitor cell 4m. A specific configuration of the sensor control unit 10 including the detection circuit unit 3 will be described later.

### (Configuration of Sensor Element 2)

In FIG. 3, the sensor element 2 has a layered element structure in which the heater unit 5 and a ceramic layer for the formation of the multiple electrochemical cells 4 are layered. The multiple electrochemical cells 4 are the pump cell 4p, the monitor cell 4m, and the sensor cell 4s, each of which is constituted of the solid electrolyte layer 11 and a pair of electrode units 41, 42 placed on the surface of the layer. The solid electrolyte layer 11 and a reference electrode 42, which is one of the pair of electrode units, are shared by the cells.

The sensor element 2 is a rectangular solid with a longitudinal direction X along the vertical direction in FIG. 3 and includes the electrochemical cells 4 arranged inside its one end that serves as the distal end (the lower end shown in the drawing). With the outside of the distal end covered with an element cover (not shown), the sensor element 2 is attached to protrude to inside an exhaust gas tube (not shown) and exposed to exhaust gas provided as measured gas. The other end of the sensor element 2 that serves as the proximal end, is positioned outside the exhaust gas tube and exposed to the atmosphere provided as reference gas.

As viewed in the layer direction of the sensor element 2, the measured gas chamber 22 is formed adjacent to a first surface of the solid electrolyte layer 11, whereas a reference gas chamber 23 is formed adjacent to the a second surface of the solid electrolyte layer 11. The measured gas chamber 22 receives the exhaust gas through the diffusion resistance layer 21 formed in the distal end surface of the sensor element 2, whereas the reference gas chamber 23 receives the atmosphere through an opening formed in the proximal end surface of the sensor element 2.

Each electrochemical cell 4 has the pair of electrode units 41, 42 placed opposite to each other on opposing sides of the shared solid electrolyte layer 11. The first surface of the solid electrolyte layer 11 in contact with the measured gas is a measuring surface, and the second surface in contact with the reference gas is a reference surface. One of the pair of electrode units 41, 42 is a pump electrode 41p included in the pump cell 4p, a monitor electrode 41m included in the monitor cell 4m, or a sensor electrode 41s included in the sensor cell 4s, and placed on the measuring surface of the solid electrolyte layer 11 facing the measured gas chamber 22. The common reference electrode 42, which is the other of the pair of electrode units 41, 42, is placed on the reference surface of the solid electrolyte layer 11 facing the reference gas chamber 23.

The solid electrolyte layer 11 is shaped as a rectangular plate, and on the surface adjacent to the measured gas chamber 22, an insulator layer 12 including the diffusion resistance layer 21 is placed and overlaid with a shield layer 13. On the surface of the solid electrolyte layer 11 adjacent to the reference gas chamber 23, an insulator layer 14 is placed and overlaid with a heater substrate layer 51 forming the heater unit 5. At the distal end of the insulator layer 12, a rectangular hollow is formed as the measured gas chamber 22, whereas a long and narrow hollow is formed in the insulator layer 14 from the distal end to the proximal end as the reference gas chamber 23. The outer surface of the sensor element 2 is covered with a porous protective layer 15.

The solid electrolyte layer 11 is a solid electrolyte sheet that conducts oxide ions. Examples of solid electrolytes conducting oxide ions include stabilized zirconia and partially stabilized zirconia. Examples of stabilizers include at least one selected from the group consisting of yttria, calcia, magnesia, scandia, ytterbia, and hafnia, and preferably, zirconia stabilized with yttria is used.

The insulator layer 12 is, for example, a sheet of electrically insulating ceramic such as alumina, and the part of the distal end chamber wall of the measured gas chamber 22 is formed from porous ceramic as the diffusion resistance layer 21 permeable to gas. The shield layer 13 is a dense sheet of electrically insulating ceramic and forms the top surface of the measured gas chamber 22 to restrict gas permeation. Each of the layers may be formed by a known sheet forming method, and materials and porosity are adjusted to achieve desired sheet properties.

The heater unit 5 includes the heater substrate layer 51 formed from electrically insulating ceramic and a heater electrode 52 embedded in the heater substrate layer 51. The heater electrode 52 is placed in correspondence with the site of the measured gas chamber 22 and energized to generate heat, enabling all the pump cell 4p, the monitor cell 4m, and the sensor cell 4s to be heated to a temperature appropriate to a detection operation (e.g., 700° C. to 800° C.).

The measured gas chamber 22 receives exhaust gas flowing in the longitudinal direction X through the diffusion resistance layer 21 forming the distal end chamber wall. In the measured gas chamber 22, the pump electrode 41p of the pump cell 4p is formed on a distal part of the solid electrolyte layer 11, that is, an upstream surface in the gas flow. The diffusion resistance layer 21 is placed near the part of the solid electrolyte layer 11 corresponding to the bottom surface of the measured gas chamber 22 and has a width equal to the width of the measured gas chamber 22. This arrangement allows the exhaust gas including NOx and oxygen to be introduced evenly across the pump electrode 41p. Downstream of the pump electrode 41p, the monitor electrode 41m of the monitor cell 4m and the sensor electrode 41s of the sensor cell 4s are arranged side by side in a line normal to the gas flow direction.

### (Principle of Detection in Sensor Element 2)

The fundamental principle of gas concentration detection in the above-described sensor element 2 will now be described.

The pump cell 4p is capable of oxygen pumping for pumping in or pumping out oxygen between the measured gas chamber 22 and the reference gas chamber 23 in response to the application of a predetermined voltage between the pump electrode 41p and the reference electrode 42 arranged with the solid electrolyte layer 11 interposed therebetween. In this oxygen pumping, oxygen (O₂) in the exhaust gas is reductively decomposed and ionized (O₂+4e⁻→2O²⁻) at the pump electrode 41p. The generated oxide ions (O²⁻) travel through the solid electrolyte layer 11 and reach the reference electrode 42. Then, oxygen is generated and discharged (2O²⁻→O₂+4e⁻) at the reference electrode 42. In this case, the exhaust gas flowing in the measured gas chamber 22 is regulated by the flow resistance of the diffusion resistance layer 21, and thus output currents from the pump cell 4p represent limiting current characteristics that depend on the oxygen concentration of the exhaust gas.

The above characteristics may be used. By setting an applied voltage so as to be in the limiting current range of oxygen, the air-fuel ratio A/F of the exhaust gas introduced into the measured gas chamber 22 can be determined based on the output current flowing in the pump cell 4p, using the atmosphere introduced into the reference gas chamber 23 as a reference. The pump cell 4p receives an AC voltage applied from the detection circuit unit 3 for simultaneous detection of the air-fuel ratio A/F and cell impedance Zac. The detection circuit unit 3 will be described later.

The pump electrode 41p, the monitor electrode 41m, and the sensor electrode 41s may be designed as porous cermet electrodes including a noble metal or a noble metal alloy, such as Pt, Au, or Rh, and having gas permeability. The pump electrode 41p of the pump cell 4p is desirably inactive for decomposition of NOx and, for example, may be a porous cermet electrode including Au—Pt or the like. This allows the NOx in the exhaust gas to reach the monitor cell 4m and the sensor cell 4s downstream from the pump cell 4p without being decomposed.

Likewise the pump electrode 41p, the monitor electrode 41m of the monitor cell 4m is desirably inactive for decomposition of NOx and, for example, may be a porous cermet electrode including Au—Pt or the like. The sensor electrode 41s of the sensor cell 4s is desirably active for decomposition of NOx and, for example, may be a porous cermet electrode including Pt or Pt—Rh or the like. The reference electrode 42 may be a porous cermet electrode including a noble metal such as Pt.

In the monitor cell 4m, when a predetermined voltage is applied between the monitor electrode 41m and the reference electrode 42, the residual oxygen in the exhaust gas is decomposed and discharged into the reference gas chamber 23, causing a limiting current to flow. In the sensor cell 4s, when the predetermined voltage is applied between the sensor electrode 41s and the reference electrode 42, oxide ions are discharged into the reference gas chamber 23 on the basis of the residual oxygen in the exhaust gas and also the oxygen resulting from the decomposition of NOx, causing a limiting current to flow. Accordingly, the output current from the monitor cell 4m may be compared with the output current from the sensor cell 4s to determine the NOx concentration of the exhaust gas.

### (Configuration of Sensor Control Unit 10)

The specific configuration of the sensor control unit 10 of the gas sensor 1 and overall sensor control will now be described. In FIG. 2, the sensor control unit 10 includes the detection circuit unit 3 provided in correspondence with at least one or two or more of the electrochemical cells 4 in the sensor element 2. The detection circuit unit 3 in this example is provided in correspondence with the pump cell 4p of the sensor element 2, and includes the AC voltage application unit 31 including the sinusoidal wave generation unit 311 as an AC voltage generation unit, the gas concentration detection unit 32 that detects the oxygen concentration (air-fuel ratio) based on AC output from the pump cell 4p, and the cell temperature detection unit 33 that detects the cell temperature.

The sensor control unit 10 further includes the heater control unit 50 that controls the operation of the heater unit 5 and the NOx detection unit 60 that detects the NOx concentration based on output from the monitor cell 4m and the sensor cell 4s. The heater control unit 50 controls the energization of the heater unit 5 so that the sensor element 2 is in a desired temperature range, based on the cell temperature information detected by the cell temperature detection unit 33. The heater unit 5 is energized through, for example, known PWM control, and the duty ratio of the battery voltage applied in the form of pulses may be varied to enable feedback control of the element temperature.

With the measured gas chamber 22 adjusted to a low oxygen concentration through oxygen pumping by the pump cell 4p, the NOx detection unit 60 applies the predetermined voltage to the sensor cell 4s and the monitor cell 4m with the reference electrode 42 being positive, and measures the limiting current flowing in each cell. In this condition, as described above, the current corresponding to the oxide ions resulting from the decomposition of the NOx and the residual oxygen flows in the sensor cell 4s, whereas the current corresponding only to the residual oxygen flows in the monitor cell 4m, thus enabling the NOx concentration to be detect based on the difference value between the measured limiting currents.

In FIG. 1, the AC voltage application unit 31 of the detection circuit unit 3 includes the sinusoidal wave generation unit 311 as an AC voltage generation unit for generating an AC voltage signal to be applied to the pump cell 4p. The sinusoidal wave generation unit 311 generates a desired sinusoidal wave signal (sin ωt) as an AC voltage signal and, for example, continuously applies the generated signal between the pump electrode 41p and the reference electrode 42 of the pump cell 4p via an amplifier 30. The resultant alternating current flowing between the electrodes of the pump cell 4p depending on the applied voltage is detected continuously and input to the gas concentration detection unit 32 as a detected signal converted from current to voltage by the current-to-voltage conversion unit 20 (hereinafter also referred to as a converted voltage signal).

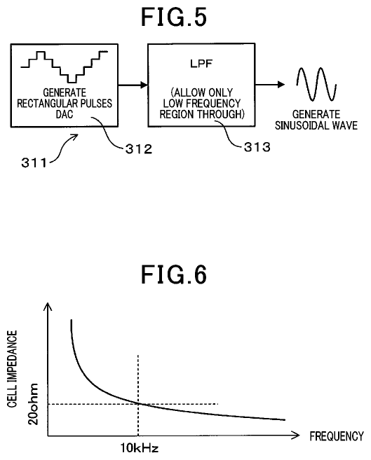

As shown in FIG. 5, the sinusoidal wave generation unit 311 may be, for example, a combination of a waveform generation unit 312 including a D/A converter (i.e., DAC in the drawing) and a low-pass filter (i.e., LPF in the drawing) 313. The waveform generation unit 312 is capable of generating an AC signal having continuous rectangular pulses and a predetermined frequency. The output signal from the waveform generation unit 312 has a waveform that changes digitally, and thus the low-pass filter 313, which allows only a low frequency region through, is used to approximate a sinusoidal wave signal. This processing enables the generation and output of a sinusoidal wave signal as illustrated in the drawing.

In this processing, as shown in FIG. 6, the frequency of the AC voltage signal fed from the AC voltage application unit 31 to the sensor element 2 is set at any level in accordance with the impedance of the electrochemical cell 4 detected by the detection circuit unit 3 (hereinafter also referred to as cell impedance). The frequency of the AC voltage signal is inversely proportional to the cell impedance, and as the frequency lowers, the cell impedance increases and changes more greatly. Thus, for example, the frequency can be consistently detected by the detection circuit unit 3 within a range in which the cell impedance varies more slightly relative to changes in the frequency, and the frequency may be appropriately set (e.g., to 10 kHz) to achieve an impedance level appropriate to detection (e.g., 20 ohms).

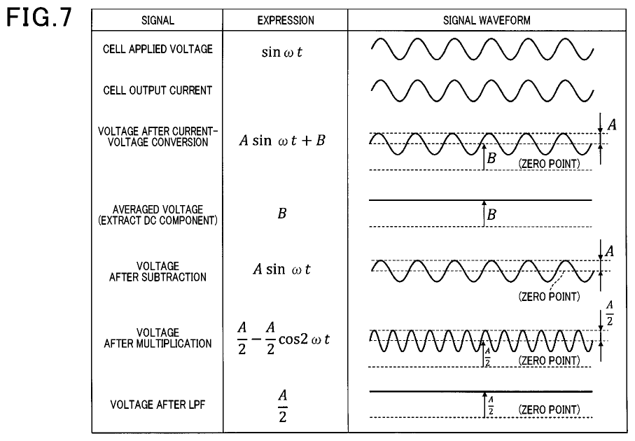

In this processing, as shown in FIG. 7, the AC voltage signal applied from the AC voltage application unit 31 to the pump cell 4p of the sensor element 2 (hereinafter also referred to as the cell applied voltage) is represented by a sinusoidal wave signal sin ωt as a signal waveform having a predetermined amplitude and a predetermined frequency. In response, the pump cell 4p of the sensor element 2 provides a cell output current corresponding to the gas concentration (i.e., the air-fuel ratio of the measured gas). The cell output current is subjected to current-to-voltage conversion at the current-voltage conversion unit 20 and then input to the gas concentration detection unit 32 as a converted voltage signal.

The converted voltage signal includes an AC signal component A sin ωt containing cell temperature information and a DC signal component B containing gas concentration information. The AC signal component A sin ωt has an amplitude A corresponding to the cell impedance (Zac) representing the cell temperature information. The level of the DC signal component B corresponds to the air-fuel ratio (A/F) representing the gas concentration information. The relationship between the signals is as follows:

Sinusoidal wave signal: sin ωt
Converted voltage signal: A sin ωt+B
A: Signal component corresponding to cell temperature information (cell impedance Zac)
B: Signal component corresponding to gas concentration information (air-fuel ratio A/F)

The AC signal component A sin ωt is a sinusoidal wave signal having the same frequency and phase as the AC voltage signal that is the cell applied voltage and having a different amplitude. The change in amplitude depends on the cell impedance Zac. Thus, the AC signal component A sin ωt may be separated from the converted voltage signal to extract a signal including the cell temperature information. Likewise, the DC signal component B may be separated from the converted voltage signal to extract a signal including the gas concentration information.

However, when the output from the sensor element 2 is affected by noise, the converted voltage signal shown in FIG. 7 becomes A sin ωt+B+(noise component). The noise component is extracted from the converted voltage signal together with the AC signal component A sin ωt. It is thus necessary to extract a signal including only the cell temperature information by separating the AC signal component A sin ωt and also eliminating the noise component. A circuit configuration designed to achieve this is described below.

In FIG. 1, the gas concentration detection unit 32 includes the averaging processing unit 321 that averages the converted voltage signal A sin ωt+B to extract the DC signal component B. The averaging processing unit 321 may be, for example, a low-pass filter shown in FIG. 2 (i.e., LPF). As a result, as shown in FIG. 7, the AC component is removed, and only the DC signal component B is output as an averaged voltage signal.

In this manner, the gas concentration detection unit 32 enables the DC signal component B including the gas concentration information to be extracted from the converted voltage signal A sin ωt+B. The DC signal component B in this example corresponds to the air-fuel ratio A/F of the exhaust gas introduced into the pump cell 4p, and is output at any time, for example, from the sensor control unit 10 to the engine ECU as an air-fuel ratio A/F signal and used for air-fuel ratio control.

In FIG. 1, the cell temperature detection unit 33 includes the signal extraction unit 34 and the synchronous detection unit 35. The signal extraction unit 34 includes the subtraction processing unit 341 and extracts the AC signal component A sin ωt from the output signal provided by the electrochemical cell 4. Specifically, as shown in FIG. 7, the subtraction processing unit 341 subtracts, from the converted voltage signal A sin ωt+B, the DC signal component B extracted in the gas concentration detection unit 32, and as a result, the AC signal component A sin ωt is extracted as a voltage signal after subtraction.

Furthermore, the synchronous detection unit 35 includes the multiplication processing unit 351 and the filter unit 352 and extracts a signal containing no noise component. Specifically, the multiplication processing unit 351 multiplies the AC signal component A sin ωt obtained through the subtraction by the sinusoidal wave signal sin ωt generated by the AC voltage application unit 31. As shown in FIG. 7 as a voltage signal after multiplication, the signal resulting from the multiplication processing is expressed as described below using a trigonometric formula.

$$sin ω t \cdot A sin ω t = ( A / 2 ) \cdot \{ cos ( 0 ) - A \cdot cos ( ω t + ω t ) / 2 \} = ( A / 2 ) - ( A / 2 ) \cdot cos ( 2 ω t )$$

The filter unit 352 may be, for example, a low-pass filter (i.e., LPF). The voltage signal after multiplication is a signal including a DC component having half the amplitude and an AC component having twice the angular frequency relative to the AC signal component A sin ω before multiplication. Thus, the noise component such as high frequency noise can be removed together with the AC component by allowing only the DC component of the signal to pass through the filter unit 352. As shown in FIG. 7 as a voltage signal after LPF, this processing can extract (A/2), which is the DC component included in the voltage signal after multiplication.

In this manner, the cell temperature information containing no noise component can be extracted in the cell temperature detection unit 33 by subtracting the DC signal component B from the converted voltage signal A sin ωt+B and then performing synchronous detection. The voltage signal after LPF (A/2) in this example corresponds to the cell impedance Zac of the pump cell 4p, and is output at any time, for example, to the heater control unit 50 of the sensor control unit 10 and used for energization control of the heater unit 5.

The averaging processing unit 321 of the gas concentration detection unit 32 may be, for example, an analog circuit (including a resistor, a capacitor, and an inductor). If designed as a digital circuit, the averaging processing unit may, for example, compute the moving average of a predetermined number of samples to extract the DC signal component B. Similarly, the low-pass filter 313 of the AC voltage application unit 31 or a low-pass filter used as the filter unit 352 of the cell temperature detection unit 33 may be an analog filter or a digital filter.

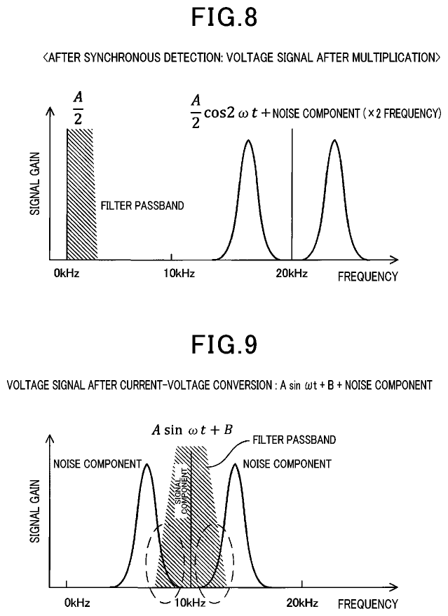

The effect of signal processing by the cell temperature detection unit 33 on a signal containing a noise component will now be described with reference to FIGS. 8 and 9. As shown in FIG. 9, when output from the sensor element 2 contains a noise component, the converted voltage signal input to the detection circuit unit 3 represents A sin ωt+B+noise component. In this case, typically, a band-pass filter is used to allow the passage of only components in a specific frequency band corresponding to the signal component in order to extract the signal component A sin ωt+B. However, when a noise component overlaps the passband of the band-pass filter (e.g., hatched in the drawings), a part of the overlapping component (e.g., regions encircled with dashed lines) passes through the filter. Additionally, band-pass filters have varying passbands due to filter constant tolerances, and thus accurate signal detection is challenging.

In contrast, as shown in FIG. 8, when synchronous detection is performed in the cell temperature detection unit 33 after the subtraction processing of the converted voltage signal, the voltage signal after multiplication having passed through the multiplication processing unit 351 is expressed as:

Voltage signal after multiplication: (A/2)−(A/2)·cos(2ωt)+noise component (×2 frequency)

This processing causes the signal component containing the cell temperature information to be DC and also doubles the frequency of the noise component, thus enabling the noise component to be removed through the filter unit 352, which allows a low frequency region through. The filter unit 352 may have any cutoff frequency (e.g., 10 kHz). In this case, the low-pass filter included in the filter unit 352 may be typically designed to be steeper than a band-pass filter and theoretically set to have a passband of frequencies as close as possible to a DC component, thus enabling accurate signal detection.

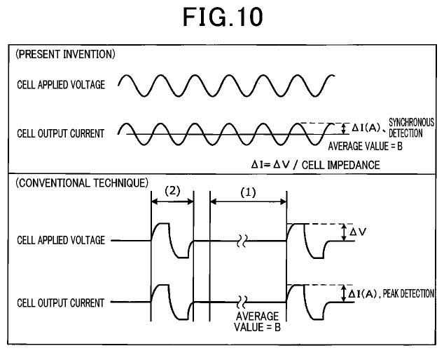

As described, in the present embodiment, an AC voltage signal is used as a cell applied voltage, and as illustrated in the upper part of FIG. 10, a cell output current can be always obtained by applying a voltage to the electrochemical cell 4 (e.g., the pump cell 4p). It is noted that the average value of the AC signal serving as the cell output current in the drawing corresponds to the DC signal component B and is a value that depends on the gas concentration information (e.g., air-fuel ratio A/F). A variation ΔI in the cell output current corresponds to the amplitude A of the AC signal component A sin ωt and is a value that depends on the cell impedance Zac serving as the cell temperature information. That is, the variation ΔI in the cell output current is obtained by dividing a variation ΔV in the cell applied voltage by the cell impedance Zac as expressed by the following equation:

ΔI=ΔV/Zac

In this process, as described above, the DC signal component B indicating the gas concentration information is extracted from the continuously output converted voltage signal A sin ωt+B of cell output currents, and simultaneously, the AC signal component A sin ωt is subjected to synchronous detection. This processing enables the cell temperature information containing no noise component to be extracted. Thus, in the present embodiment, the gas concentration information and the cell temperature information can be obtained in real time with high accuracy, improving the control efficiency of the sensor control unit 10 or the engine ECU.

In contrast, a conventional technique shown in the lower part of FIG. 10 involves switching between a period (1) during which a DC voltage signal is output as a cell applied voltage and a period (2) during which an AC voltage signal is output, and simultaneous detection cannot be performed. In this case, for example, a DC voltage for gas concentration detection is applied during the period (1), and a DC output current is detected. Then, an AC voltage signal for cell temperature detection is applied during the period (2), and the variation ΔI in the cell output current is detected based on the peak current value of the AC output current. For this reason, a change in the gas concentration information or the cell temperature information cannot be quickly detected, causing a delay in control. Moreover, the noise component cannot be eliminated from the cell temperature information and may degrade the detection accuracy.

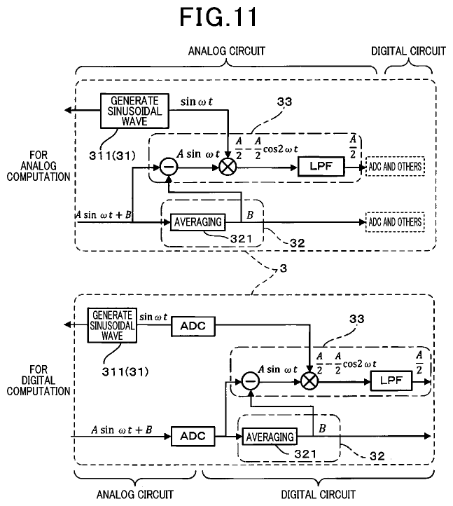

As shown in FIG. 11, the detection circuit unit 3 has a main circuit part for computation, which may be an analog circuit or a digital circuit. For analog computation shown in the upper part of FIG. 11, a digital circuit including A/D converters (i.e., ADC in the drawing) is placed near the outputs of the gas concentration detection unit 32 and the cell temperature detection unit 33 and externally outputs converted signals in digital form.

For digital computation shown in the lower part of FIG. 11, A/D converters are placed between the sensor element 2 and the AC voltage application unit 31, and the gas concentration detection unit 32 and the cell temperature detection unit 33, and input converted signals in digital form. The gas concentration detection unit 32 and the cell temperature detection unit 33 are digital circuits such as microcomputers and externally output detected signals.

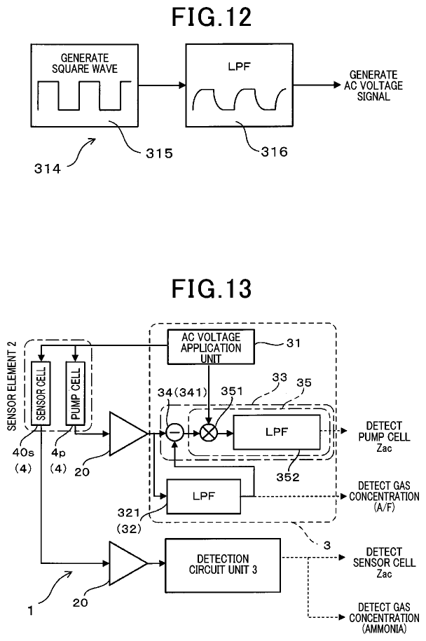

As shown in FIG. 12, the AC voltage application unit 31 in the detection circuit unit 3 may apply not a sinusoidal wave signal but an AC voltage signal generated from a square wave signal. In this case, in place of the sinusoidal wave generation unit 311, the AC voltage application unit 31 includes an AC voltage generation unit 314 having a combination of a waveform generation unit 315 and a low-pass filter (i.e., LPF in the drawing) 316. The waveform generation unit 315 generates a square wave continuing with a predetermined period and passes an output signal through the low-pass filter 316 to give an AC voltage signal having a smooth waveform. Also in this case, gas concentration information and cell temperature information may be obtained based on an output signal from the sensor element 2.

### Second Embodiment

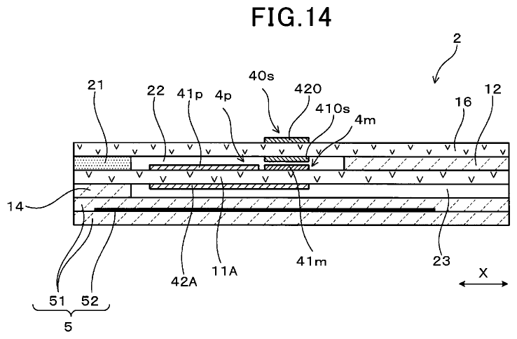

A gas sensor according to a second embodiment will now be described with reference to FIGS. 13 and 14. In the present embodiment, a gas sensor 1 is a limiting current ammonia sensor with a detection circuit unit 3 provided for each of multiple electrochemical cells 4 in a sensor element 2 to enable detection of the gas concentration and the cell temperature. The sensor element 2 and a sensor control unit 10 have the same basic configurations as in the first embodiment, and differences from the first embodiment will be mainly described.

In the second and subsequent embodiments, the same reference signs as used in a previous embodiment indicate the same components as described in the previous embodiment, unless otherwise noted.

As shown in FIG. 13, the gas sensor 1 in the present embodiment includes a detection circuit unit 3 connected to the pump cell 4p of the sensor element 2 and also a detection circuit unit 3 provided for a sensor cell 40s that detects ammonia. The detection circuit unit 3 connected to the sensor cell 40s is not illustrated because it has the same configuration as the detection circuit unit 3 connected to the pump cell 4p. The sensor control unit including the detection circuit units 3 is similarly not illustrated. Although the sensor element 2 may include a monitor cell 4m as described below, the monitor cell may not be included.

As shown in FIG. 14, the sensor element 2 in the present embodiment includes multiple solid electrolyte layers 11A and 16. The first solid electrolyte layer 11A conducting oxide ions has a measured gas chamber 22 and a reference gas chamber 23 on opposing sides of the layer. The reference gas chamber 23 receives a first reference gas, such as the atmosphere. In the measured gas chamber 22, into which exhaust gas is introduced through the diffusion resistance layer 21, the pump electrode 41p included in the pump cell 4p and the monitor electrode 41m included in the monitor cell 4m are arranged on the surface of the first solid electrolyte layer 11A. On the surface of the first solid electrolyte layer 11A facing the reference gas chamber 23, a first reference electrode 42A is placed opposite the pump electrode 41p and the monitor electrode 41m. Furthermore, the reference gas chamber 23 is defined by an insulator layer 14, on which a heater unit 5 is superposed.

In the present embodiment, the sensor element 2 includes the sensor cell 40s for ammonia detection in place of the sensor cell 4s for NOx detection in the first embodiment. Accordingly, in place of the shield layer 13 in the first embodiment, the second solid electrolyte layer 16 conducting protons is superposed on the insulator layer 12 forming the side surface of the measured gas chamber 22. A first surface of the second solid electrolyte layer 16 is the top surface of the measured gas chamber 22 and serves as a measuring surface in contact with measured gas, and a sensor electrode 410s for detecting ammonia as a specific gas is placed on the surface. A second surface of the second solid electrolyte layer 16 serves as a reference surface, and a second reference electrode 420 in contact with a second reference gas is placed on the surface.

The second reference gas for the sensor cell 40s for ammonia detection may be the first reference gas, such as the atmosphere also used in NOx detection, or a gas of the same kind as measured gas such as the exhaust gas. In the present embodiment, with the reference surface of the second solid electrolyte layer 16 exposed outside, the reference electrode 420 of the sensor cell 40s is exposed to the measured gas serving as the second reference gas. The outer surface of the sensor element 2 may be covered with the same protective layer 15 as in the first embodiment, or a reference gas chamber that receives the measured gas may be further formed outside the reference electrode 420 to protect the second the reference electrode 420.

The sensor electrode 410s is provided in a manner to face the monitor electrode 41m, and the sensor electrode 410s and the monitor electrode 41m are arranged in a line normal to the gas flow direction. The reference electrode 420 is positioned on the opposite side of the solid electrolyte layer 16 from the sensor electrode 410s and is exposed outside. In this arrangement, when the sensor electrode 410s and the reference electrode 420 are exposed to the same kind of gas atmosphere, a potential shift that may occur between the electrodes due to gas atmosphere difference may be prevented, reducing the error between potentials at which limiting currents are produced.

The proton-conducting solid electrolyte that forms the solid electrolyte layer 16 is preferably composed of a perovskite-type oxide. Examples of perovskite-type oxides include, but are not limited to, strontium zirconate, calcium zirconate, barium zirconate, strontium cerate, calcium cerate, and barium cerate, which are doped with a rare earth element such as Y or Yb. The solid electrolyte layer 16 may contain at least one of the perovskite-type oxides.

The sensor electrode 410s is a porous cermet electrode including a noble metal or a noble metal alloy such as Pt and may suitably contain an acidic substance that increases the property of adsorbing ammonia, which is a base. Examples of acidic substances include phosphate compounds such as phosphate and pyrophosphate.

The fundamental principle of gas concentration detection performed by the gas sensor 1 having the above configuration will now be described.

Also in this embodiment, the exhaust gas introduced into the measured gas chamber 22 is adjusted to a predetermined low oxygen concentration by the pump cell 4p before reaching the downstream sensor cell 40s and monitor cell 4m. In the monitor cell 4m, the decomposition of the residual oxygen in the exhaust gas causes a limiting current to flow, allowing the residual oxygen concentration to be monitored. Alternatively, the electromotive forces of both the electrodes may be detected. Based on the detection results from the monitor cell 4m, the pump cell 4p is controlled to maintain a sufficiently low oxygen concentration level in the measured gas chamber 22, enabling a reduction in the effect of the residual oxygen in the sensor cell 40s.

In the sensor cell 40s, the decomposition reaction of ammonia contained in the exhaust gas having reached the sensor electrode 410s generates protons (H⁺). The reaction formula is as follows:

2NH₃→6H⁺+6e⁻

The generated protons pass through the solid electrolyte layer 16 and reach the reference electrode 420. In the reference electrode 420, the protons react with oxygen to produce water. The reaction formula is as follows:

6H⁺+3/2O₂+6e⁻→3H₂O

When these reactions in the sensor cell 40s progress smoothly, the supply of ammonia is regulated by the diffusion resistance layer 21, and thus the diffusion of ammonia to the sensor electrode 410s is a rate determining step. Therefore, a limiting current dependent on the ammonia concentration of the exhaust gas flows between the sensor electrode 410s and the reference electrode 420. Based on the limiting current, the ammonia concentration may be detected.

It is desirable that the sensor cell 40s be controlled to a temperature that is equal to or higher than an operating temperature appropriate to ammonia detection and is low relative to the pump cell 4p and the monitor cell 4m (e.g., about 400° C. to 600° C.). For example, it has been found that, when the sensor cell 40s is at 350° C., and exhaust gas with an oxygen concentration greater than 300 ppm reaches the sensor electrode 410s, the solid electrolyte layer 16 may conduct electrons produced by the decomposition of oxygen. In contrast, in the pump cell 4p and the monitor cell 4m, as the temperature increases, oxygen is ionized more efficiently and discharged more readily.

For this reason, the sensor cell 40s has the sensor electrode 410s placed farther away from the heater unit 5 than the pump electrode 41p and the monitor electrode 41m. In addition, since the solid electrolyte layer 16 on which the sensor electrode 410s is provided is exposed outside, the temperature rise can be easily suppressed. Furthermore, the detection circuit unit 3 is provided for each of the pump cell 4p and the sensor cell 40s, enabling independent control of each temperature.

In FIG. 13, the detection circuit unit 3 connected to the pump cell 4p has the same configuration and operation as in the first embodiment described above, and includes an AC voltage application unit 31, a gas concentration detection unit 32 that detects the air-fuel ratio A/F as gas concentration information, and a cell temperature detection unit 33 that detects the cell impedance Zac of the pump cell 4p as temperature information.

The detection circuit unit 3 connected to the sensor cell 40s also includes the same AC voltage application unit 31, gas concentration detection unit 32, and cell temperature detection unit 33. The gas concentration detection unit 32 detects the ammonia concentration as gas concentration information, and the cell temperature detection unit 33 detects the cell impedance Zac of the sensor cell 40s as temperature information.

In this processing, the gas concentration and the temperature are detected from each of the pump cell 4p and the sensor cell 4s, thus enabling the heater control unit 50 to be operated to achieve an optimum temperature for each cell based on its individual detection results. In this case, the temperature of the sensor cell 4s dependent on the energization of the heater electrode 52 in the heater unit 5 can be controlled based on actual measurements, thus preventing the controlled temperature from being out of a desired temperature range and enabling the gas concentration to be detected with higher accuracy.

The present disclosure is not limited to the embodiments described above, but applicable to various embodiments without departing from the spirit and scope thereof. For example, in the above embodiments, the gas sensor 1 determines the exhaust gas from the vehicle engine as measured gas, and the sensor element 2 detects oxygen, NOx, or ammonia contained in the exhaust gas. However, other gases contained in the exhaust gas may also be detected.

Additionally, in the above embodiment, the sensor element 2 has a triple-cell structure, and the detection circuit unit 3 is provided for one or two of its electrochemical cells 4. However, the sensor element 2 may have a single-cell or a dual-cell structure, or the detection circuit unit 3 may be connected to every cell of the electrochemical cells 4. Moreover, the measured gas may not be the exhaust gas from a vehicle engine but may be exhaust and other gases from various internal combustion engines.

Although the present disclosure has been described in accordance with the embodiments, it will be understood that the disclosure is not limited to the embodiments or the structures. This disclosure encompasses various modifications and alterations falling within the range of equivalence. Additionally, various combinations and forms as well as other combinations and forms with one, more than one, or less than one of the elements added thereto also fall within the scope and spirit of the present disclosure.

What is claimed is:

1. A gas sensor comprising:
a sensor element; and
a detection circuit unit configured to detect a specific gas component in measured gas based on output from the sensor element,
wherein the sensor element includes
a measured gas chamber configured to receive the measured gas through a diffusion resistance layer, and
an electrochemical cell including a pair of electrode units respectively provided on a first surface and a second surface of a solid electrolyte layer, the first surface being in contact with the measured gas and the second surface being in contact with reference gas,
the detection circuit unit includes
an AC voltage application unit configured to apply an AC voltage signal to the electrochemical cell,
a gas concentration detection unit including an averaging processing unit configured to average an output signal provided by the electrochemical cell to extract a DC signal component included in the output signal provided by the electrochemical cell, and configured to detect concentration information on the specific gas component from the DC signal component, and
a cell temperature detection unit configured to detect temperature information on the electrochemical cell from an AC signal component included in the output signal provided by the electrochemical cell, and
the cell temperature detection unit includes
a signal extraction unit including a subtraction processing unit configured to subtract the extracted DC signal component from the output signal provided by the electrochemical cell to extract the AC signal component, and
a synchronous detection unit configured to perform synchronous detection on the extracted AC signal component using the AC voltage signal, and
the synchronous detection unit includes a multiplication processing unit configured to multiply the extracted AC signal component by the applied AC voltage signal, and a filter unit configured to filter the signal after the multiplication to allow passage of components with frequencies lower than a frequency of the applied AC voltage signal.

2. The gas sensor according to claim 1, wherein
the AC voltage application unit includes an AC voltage generation unit configured to generate a sinusoidal wave signal or a rectangular wave signal as the AC voltage signal, and is configured to continuously apply the AC voltage signal.

3. The gas sensor according to claim 1, wherein
the sensor element includes a plurality of the electrochemical cells, and
the detection circuit unit is provided in correspondence with one or more of the electrochemical cells.

4. The gas sensor according to claim 3, further comprising:
a heater control unit configured to control operation of a heater unit incorporated in the sensor element,
wherein the heater control unit is configured to perform feedback control of a temperature of the sensor element based on a detection result from the cell temperature detection unit included in the one or more detection circuit units.

5. The gas sensor according to claim 1, wherein
the sensor element has a reference gas chamber configured to receive reference gas,
the sensor element includes, as the electrochemical cells,
a pump cell including a pump electrode placed, near the diffusion resistance layer, on a first surface of the solid electrolyte layer facing the measured gas chamber, and a reference electrode placed, opposite the pump electrode, on a second surface of the solid electrolyte layer facing the reference gas chamber, and
a sensor cell including a sensor electrode placed, downstream of the pump electrode, on the first surface of the solid electrolyte layer, and a reference electrode placed, opposite the sensor electrode, on the second surface of the solid electrolyte layer, and
at least the pump cell is provided with the detection circuit unit.

6. The gas sensor according to claim 1, wherein
the sensor element has a reference gas chamber configured to receive a first reference gas,
the sensor element includes, as the electrochemical cells,
a pump cell including a pump electrode placed, near the diffusion resistance layer, on a first surface of a first solid electrolyte layer serving as the solid electrolyte layer, the first surface facing the measured gas chamber, and a first reference electrode placed, opposite the pump electrode, on a second surface of the first solid electrolyte layer facing the reference gas chamber,
a sensor cell including a second solid electrolyte layer facing the first solid electrolyte layer across a space serving as the measured gas chamber, a sensor electrode placed, downstream of the pump electrode, on a first surface of the second solid electrolyte layer facing the measured gas chamber, and a second reference electrode placed, opposite the sensor electrode, on a second surface of the second solid electrolyte layer in contact with a second reference gas, and
at least one of the pump cell and the sensor cell is provided with the detection circuit unit.

7. The gas sensor according to claim 6, wherein
the second reference gas is a gas of the same kind as the first reference gas or a gas of the same kind as the measured gas.
